(19) United States
(12) Patent Application Publication
(10) Pub. No.: US 2021/0059582 A1
(43) Pub. Date: Mar. 4, 2021

(54) Non-Invasive Glucose Monitoring by Raman Spectroscopy

(71) Applicants: Jeon Woong Kang, Melrose, MA (US); Peter T. C. So, Boston, MA (US)

(72) Inventors: Jeon Woong Kang, Melrose, MA (US); Peter T. C. So, Boston, MA (US)

(21) Appl. No.: 16/914,785

(22) Filed: Jun. 29, 2020

Related U.S. Application Data
(60) Provisional application No. 62/893,902, filed on Aug. 30, 2019.

(51) Int. Cl. A61B 5/145; A61B 5/00

(52) U.S. Cl. CPC A61B 5/14532; A61B 5/7275; A61B 5/0075

(57) ABSTRACT

Noninvasive glucose monitoring has been a long-standing need in diabetes management. Among many approaches to meeting this need, Raman spectroscopy has attracted attention due to its molecular specificity. Previous Raman-based glucose sensing can predict blood glucose concentration based on a statistical correlation between the reference glucose concentration and unspecified spectral features. However, the lack of glucose Raman peaks and non-prospective prediction have led to questions about the effectiveness of in vivo Raman spectroscopy for transcutaneous glucose sensing. Here, we disclose technology for directly observing distinct glucose Raman spectra from skin. The Raman signal intensities were proportional to the reference glucose concentrations in three live swine glucose clamping experiments. Tracking the spectral intensity based on the linearity enables prospective prediction with high accuracy in within-subject and inter-subject models. Compared to previous statistical approaches, prospective predictions based on a direct glucose signal from the skin offers robust, reliable noninvasive glucose sensing.

### CROSS-REFERENCE TO RELATED APPLICATION(S)

This application claims the priority benefit, under 35 U.S.C. 119(e), of U.S. Application No. 62/893,902, which was filed on Aug. 30, 2019, and is incorporated herein by reference in its entirety.

### GOVERNMENT SUPPORT

This invention was made with Government support under Grant No. P41 EB015871 awarded by the National Institutes of Health (NIH). The Government has certain rights in the invention.

### BACKGROUND

Monitoring blood glucose is of importance considering the increasing population of diabetics and the associated costs of treating them. However, the painful lancing process for obtaining blood drops by finger-stick hinders people from actively monitoring blood glucose levels. Several studies have reported that more than half of the Type 1 diabetes patients do not perform daily self-monitoring, even though it is highly recommended in order to avoid the risk of various complications, such as cardiovascular diseases, ketoacidosis, and renal failure. Given this lack of compliance, reliable noninvasive blood glucose monitoring has been highly desired to provide people in need with pain-free, convenient, and continuous or frequent blood glucose measurements.

Over the past decades, a variety of noninvasive blood glucose monitoring technologies have been pursued. Among many, optical spectroscopic methods have attracted a fair amount of attention. While near-infrared (NIR) absorption spectroscopy has demonstrated some potential, extracting glucose-specific features in the presence of many confounding signals from in vivo NIR absorption spectroscopy measurements has been challenging. The NIR absorption features of glucose in the overtone and combination bands are broad and interfere with the absorption of other chromophores in tissue. Moreover, other noise factors, such as changes in temperature and contact pressure, easily dominate weak glucose signals in in vivo experiments.

Raman spectroscopy has been recognized as another promising method of noninvasive blood glucose monitoring. Raman spectra have distinctive spectral features, specific for target molecules, including glucose. Quantitative analysis for diagnostic feasibility has been reported using various biological samples such as serum, blood, tissue, and skin.

Multiple Raman instruments have been developed and tested for glucose monitoring in vivo. For example, a free-space Raman spectroscopy system collects a Raman signal from a human forearm using paraboloidal mirror combined with an f/1.8 spectrograph and a tall detector in a reflection geometry. However, its in-line geometry admits unwanted Rayleigh light reflected from the tissue surface. The free-space tissue interface is also prone to the subject movement. In another example, a transmission Raman instrument with a non-imaging optical element harvests most Raman photons emerging from the tissue. A compound hyperbolic concentrator at the tissue interface effectively collects Raman photons from a large solid angle. A transmission measurement from the thenar fold uses a contact interface, which pinches the tissue and changes its properties during the long-term measurement. More recently, an optical fiber probe-based Raman instrument with a custom-designed tissue interface reliably measured the Raman signal from the same tissue spot under room light. However, the focused radiance of the laser beam from one excitation fiber limits the sampling volume. And the small tip of the Raman probe presses the skin over hours of measurement, which might prevent glucose-containing interstitial fluid (ISF) from circulating across the sampling volume. It is common to observe a pressure mark on soft samples after using this type of probe.

For in vivo transdermal Raman spectroscopy, the acquired Raman spectra contain information of glucose molecules from ISF underneath the epidermis. High-throughput Raman spectroscopic instruments have been developed and validated with small-scale clinical trials of the human oral glucose tolerance test (OGTT) or animal glucose clamping test. Although these reports have claimed diagnostic capability with a Raman system optimized for transcutaneous measurement, they lack the characteristic Raman peaks and do not predict glucose levels. Furthermore, glucose-specific peaks in in vivo Raman spectra are very weak, subdued by strong and time-varying skin autofluorescence and associated shot noise, which make it difficult to construct good predictive models and may lead to misinterpretation of experimental results depending on the choice of validation methods.

Recent results from a glucose clamping test with a dog as subject using Raman spectroscopy purport to show measurement of a real glucose signal. These results demonstrate the similarity between the regression b-vector of the partial least squares (PLS) algorithm and the known Raman spectrum of a glucose solution, but without presenting glucose-specific Raman peaks in the measured spectra. Considering the possibility of chance correlation in a small amount of data, without firm evidence of the glucose-specific Raman peaks, there results could be inconclusive and unsuitable for prospective prediction.

### SUMMARY

Non-invasive monitoring of a blood glucose level of a mammal can be accomplished using the Raman spectroscopy methods and systems disclosed here. In some of these methods, a first Raman spectrum is acquired from an area on the mammal's skin over a first period and a second Raman spectrum is acquired from the area on the mammal's skin over a second period after the first period. A difference between the first Raman spectrum and the second Raman spectrum is determined and used to estimate a change in the blood glucose level of the mammal between the first Raman spectrum and the second Raman spectrum.

Acquiring the first Raman spectrum may involve illuminating a spot on the mammal's skin laterally displaced from the area through which the Raman spectra are acquired with a Raman pump beam forming an oblique angle with the mammal's skin. The Raman spectra are acquired by detecting Raman light scattered through the area on the mammal's skin. The oblique angle can be about 15 degrees to about 45 degrees from the Raman pump beam to the mammal's skin. The area on the mammal's skin can be laterally displaced from the spot illuminated by the laser beam by up to about 3 millimeters (e.g., 0.5, 1.0, 1.5, 2.0, or 2.5 millimeters). Detecting the Raman light may involve integrating the Raman light over the first period with a detector.

Determining the difference between the first Raman spectrum and the second Raman spectrum can include determining a difference Raman spectrum. It can also or alternatively include determining a difference in an amplitude of a peak appearing in the first Raman spectrum and the second Raman spectrum. If desired, the difference between the first Raman spectrum and the second Raman spectrum can be used to estimate a rate of change of the blood glucose level of the mammal. This rate of change can be used to predict a future blood glucose level of the mammal.

A system for non-invasively monitoring a blood glucose level of a mammal may include a Raman pump source, collection optics, and a detector array in optical communication with the collection optics. In operation, the Raman pump source illuminates a spot on the mammal's skin with a Raman pump beam incident on the mammal's skin at an oblique angle. The collection optics collects Raman light scattered through an area of the mammal's skin laterally displaced from the spot illuminated by the Raman pump beam. The detector array detects the Raman light, which represents the blood glucose level of the mammal.

The collection optics may include a fiber bundle having a distal end disposed about 3 millimeters to about 5 millimeters from the mammal's skin and proximal end in optical communication with the detector array. The distal end may be laterally displaced from the spot illuminated by the Raman pump beam by up to about 3 millimeters.

The detector array may be a two-dimensional detector array comprising at least one row for each fiber in the fiber bundle. In such a case, the system may also include a dispersive element, in optical communication with the proximal end of the fiber bundle and the two-dimensional detector array, to spectrally disperse the Raman light from each fiber along a corresponding row in the two-dimensional detector array. A filter, in optical communication with the collection optics, may transmit the Raman light to the detector array and block light at a wavelength of the Raman pump beam from the detector array.

The detector array can integrate the Raman light over a series of sequential integration periods. The system may also include a processor, operably coupled to the detector, to determine at least one difference spectrum based on the Raman light integrated by the detector array over the series of sequential integration periods and to estimate a change in the blood glucose level over at least one of the series of sequential integration periods based on the at least one difference spectrum. The processor can estimate a rate of change of the blood glucose level based on the difference spectrum.

An inventive method of non-invasively monitoring a blood glucose level of a person includes illuminating an elliptical spot on the person's skin with a Raman probe beam forming an angle of about 15 degrees to about 45 degrees with the person's skin. The distal end of a fiber bundle collects Raman light transmitted through a portion of the person's skin up to about 3 millimeters from the elliptical spot. A prism, grating, or other dispersive element spectrally disperses the Raman light from a proximal end of each fiber in the fiber bundle onto a corresponding row of detector elements in a two-dimensional detector array. The two-dimensional detector array integrates Raman spectra from the fiber bundle over a series of sequential integration periods. A processor or other device determines difference spectra based on the Raman spectra; and uses those difference spectra to estimate a rate of change in the blood glucose level of the person. The processor may also linearly extrapolate a future blood glucose level of the person based on the rate of change in the blood glucose level of the person.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1 shows a Raman spectroscopy system for measuring Raman spectra and non-invasively monitoring and estimating blood glucose levels from the Raman spectra distinguished by oblique illumination and laterally offset detection.

FIG. 2A shows a Raman pump source used in the Raman spectroscopy system shown in FIG. 1.

FIG. 2B shows a side view of a fiber bundle used as the collection optics in the Raman spectroscopy system in FIG. 1.

FIG. 2C shows a top view of a fiber bundle used as the collection optics suitable for use in the Raman spectroscopy system in FIG. 1.

FIG. 2D shows a side view of a detector in the Raman spectroscopy system in FIG. 1.

FIG. 3A illustrates an off-axis Raman excitation and collection configuration like the one used in the Raman spectroscopy system of FIG. 1.

FIG. 3B illustrates an on-axis Raman excitation and collection configuration.

FIG. 4 illustrates a method of monitoring and predicting blood glucose levels.

FIG. 5A illustrates non-invasively monitoring and predicting a blood glucose level of a person using the Raman spectroscopy system in FIG. 1.

FIG. 5B illustrates the method of FIG. 5A of non-invasively monitoring and predicting a blood glucose level of a person using the Raman spectroscopy system in FIG. 1.

FIG. 6 illustrates glucose clamping experiments with live pigs using a system like the one shown in FIG. 1.

FIG. 7 shows experimental results of fractional of sampling voxels for the off-axis configuration of FIG. 3A and the on-axis configuration of FIG. 3B, respectively, as a function of depth in the confined region.

FIG. 8A are experimental results of four glucose Raman spectra with four glucose differences from in vivo experiments and a reference Raman spectrum from a pure glucose solution.

FIG. 8B shows the linear relationships between the change in the Raman peak's intensity and the corresponding changes in glucose concentration for three different actual glucose concentration ranges over the entire recording time.

FIG. 8C shows a prediction by simple linear regression based on the peak intensity change.

FIG. 9 shows four averaged experimentally acquired spectra for four clamping periods with different average glucose concentrations.

FIG. 10A shows actual and predicted glucose concentrations versus time using PLS regression with full-range background-subtracted spectra.

FIG. 10B shows the linearity between the Raman peak intensity and glucose concentration for Trial 3.

FIG. 11 shows the glucose profile during a glucose clamping experiment in Trial 1.

FIG. 12A shows the raw spectra during the period when fluorescence stayed relatively flat.

FIG. 12B shows the glucose concentrations from an elapsed time of 345 min to an elapsed time of 250 min plotted in a time-reversed manner.

FIG. 12C shows the glucose concentration differences depending on the time difference from the reference at 345 min.

FIG. 12D shows the change in the squared Pearson correlation coefficient between the subtraction spectra and the spectrum of pure glucose in solution.

FIG. 12E shows the squared Pearson correlation coefficients as a function of glucose concentration difference.

FIG. 13 shows the estimation on the limit of detection (LoD) using a linear regression.

FIG. 14A shows the change in glucose concentrations measured in Trial 1.

FIG. 14B shows the change in glucose concentrations measured in Trial 2.

FIG. 14C shows the change in glucose concentrations measured in Trial 3.

FIG. 15A shows a glucose concentration prediction using a partial least square regression (PLSR) analysis with full-range background-subtracted spectra in Trial 1.

FIG. 15B shows Raman shifts for the PLSR b-vector of FIG. 15A and a glucose solution.

FIG. 16 shows results from Trials 1-3 in a four-fold cross-validation manner with single-subject recordings (top rows) and in a leave-one-subject-out cross-validation manner with multiple-subject recordings (bottom rows).

FIG. 17 shows the change in glucose concentration measured during an in vivo glucose clamping experiment using the Raman spectroscopy system in FIG. 1 as it compares to measurements taken with three commercial glucose meters.

FIG. 18A shows a Clarke's Error Grid Analysis of the accuracy of the Raman spectroscopy system in measuring blood glucose values during an in vivo glucose clamping experiment.

FIG. 18B shows a Clarke's Error Grid Analysis of the accuracy of an Accu-Chek blood glucose meter in measuring blood glucose values during an in vivo glucose clamping experiment.

FIG. 18C shows a Clarke's Error Grid Analysis of the accuracy of a Dexcom G6 blood glucose meter in measuring blood glucose values during an in vivo glucose clamping experiment.

FIG. 19 shows statistical results from three trials of glucose clamping experiments in vivo using the Raman spectroscopy system in FIG. 1.

### DETAILED DESCRIPTION

An off-axis Raman instrument addresses limitations of previous instruments and can directly obtain glucose Raman peaks for noninvasive blood glucose monitoring in humans, livestock, and other mammals. This off-axis Raman instrument increases or maximizes the effective sampling volume while performing a non-contact stable long-term measurement. To investigate the benefits of the particular approach(es) in this application, computations show how much volume is sampled for Raman scattered light and what fraction of total laser illumination contributes to Raman collection from a certain depth of skin tissue.

In addition to disclosing an off-axis Raman spectroscopy system for non-invasive glucose monitoring, this application discloses methods of monitoring and predicting blood glucose levels as well as results and analyses of direct observation of glucose-specific Raman peaks. The methods predict glucose concentration by taking both glucose Raman peaks and other Raman peaks related to skin components into account. Prediction of glucose levels is investigated in single and multiple subject recordings. The approach is compared to a PLSR analysis, which has been used to estimate blood glucose levels in previous studies.

The experimental data presented in this application were obtained in three swine glucose clamping experiments and may finalize the long debate about whether real glucose Raman peaks can be measured in vivo. Throughout the three trials, Raman spectra were measured from pig ears with a high optical-throughput Raman system using oblique-angle (off-axis) laser illumination. The measured spectra confirm the presence of a glucose signal and linearity between the glucose Raman peak intensities and the reference glucose concentration. The experiments allow a wide range of glucose concentrations and long integration times to obtain Raman spectra. The clamped glucose concentrations are carefully controlled by infusing dextrose solution and insulin into the swine subjects.

Off-Axis Raman Spectroscopy Systems for Non-Invasive Glucose Monitoring

A non-contact, off-axis or oblique-angle Raman spectroscopy system can identify glucose fingerprint peaks as well as observe linear changes in the corresponding glucose levels transcutaneously. This is enabled by an illumination-collection geometry to control the size and location of the sampling volume. The non-contact, off-axis Raman spectroscopy system reduces or mitigates the instability of a probe by illuminating a relatively large volume of tissue under a large fiber bundle that collects the Raman light. The off-axis illumination and vertical collection geometry of the fiber bundle spatially filters out the specular Rayleigh reflection from the skin surface, reducing the filtering burden of a Rayleigh rejection filter at the probe tip. Also, the non-contact measurement is free from potential distortion by the tissue, which is beneficial for a stable long-term measurement.

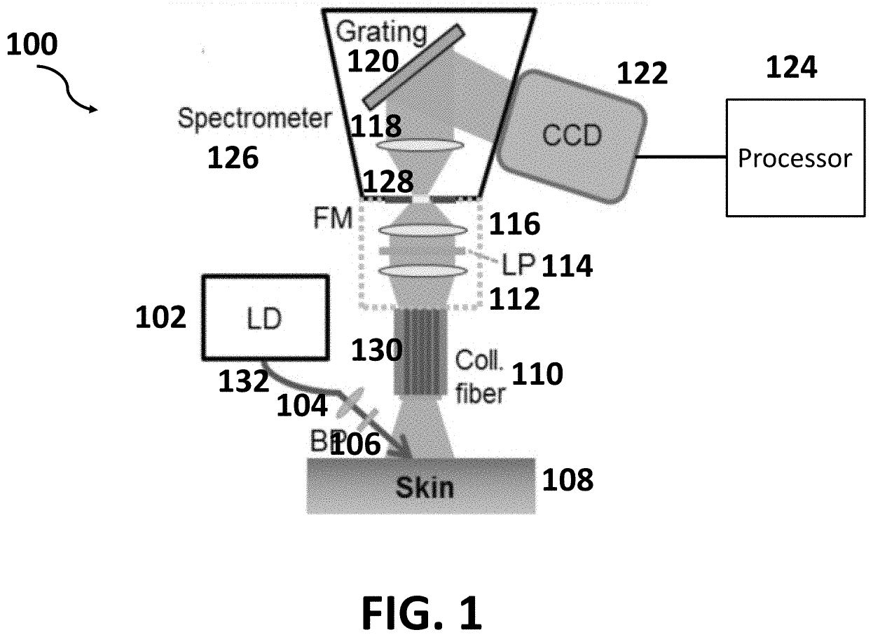

FIG. 1 shows a schematic diagram of the non-contact, off-axis Raman spectroscopy system 100 for measuring Raman spectra and non-invasively monitoring and estimating blood glucose levels from the Raman spectra distinguished by oblique illumination and laterally offset detection. The portable Raman spectroscopy instrument 100 comprises a Raman pump source 102, such as a diode laser (e.g., a 830 nm diode laser, PI-EC-830-500-FC, Process Instruments, UT USA), collection optics 110, an imaging spectrometer or spectrograph 126 (e.g., LS785, Princeton Instruments, NJ USA), and a detector array—here, a charge-coupled device (CCD) 122 (e.g., PIXIS1024BRX, Princeton Instruments, N.J. USA). The illumination-collection geometry using oblique-angle (off-axis) incidence of laser and non-contact and the Raman collection is vertical to increase the effective sampling volume of the targeted layer of the patient's skin while reducing the collection of background signals.

In operation, the Raman pump source 102 emits a Raman pump beam (e.g., at a power of 250 mW and a NIR wavelength, such as 830 nm) that propagates through and out of a probe fiber 132 and passes through a first lens 104 and a filter 106 (e.g., a band-pass filter), then illuminates an elliptical spot on the surface of a patient's skin 108 at an oblique angle. This oblique angle is about 15 degrees to about 45 degrees (e.g., about 30 degrees) from the Raman pump beam to the skin 108. (Equivalently, the Raman pump beam forms an angle of about 45 degrees to 75 degrees (e.g., about 60 degrees) with respect to the surface normal of the skin 108).

The illumination by the Raman pump beam produces a Raman signal scattered from within the skin tissue. The Raman signal is in the glucose fingerprint region (about 0-1800 cm⁻¹). For 830 nm Raman excitation, this glucose fingerprint region corresponds to a wavelength range of 830-976 nm. This Raman signal propagates through an area of the skin laterally displaced from the illuminated spot by up to about 3 millimeters. Collection optics 110 comprising a fiber bundle 130 collect the scattered Raman light. The fiber bundle 130 is attached to a second lens 112 and a long-pass filter 114 (e.g., a custom long-pass filter, Alluxa, Calif. USA) that rejects Rayleigh light. A third lens 116 is used to focus the filtered light into a spectrometer 126 that comprises a fourth lens 118, a grating 120, and the detector array (CCD) 122 for detection and further analysis. A mechanical shutter 128 installed inside of the spectrometer housing reduces vertical pixel smearing. More specifically, the shutter 128 blocks light for illuminating the CCD 122 during analog-to-digital conversion, preventing line artifacts from appearing in the CCD image.

A processor 124 is operably coupled to the detector array 122. The processor 124 is configured to determine at least one difference spectrum based on the Raman light integrated by the detector array 122 over a series of sequential integration periods. It can estimate a change in the blood glucose level over at least one of the series of sequential integration periods based on the difference spectrum. And it can estimate a rate of change of the blood glucose level based on the difference spectrum.

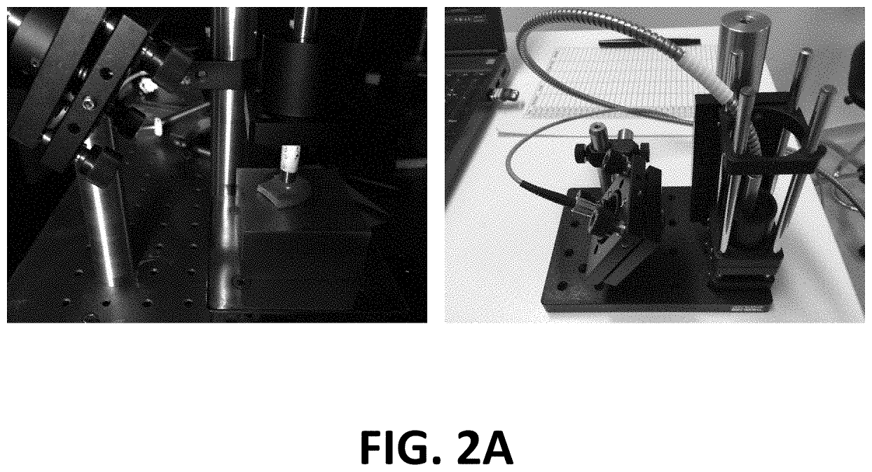

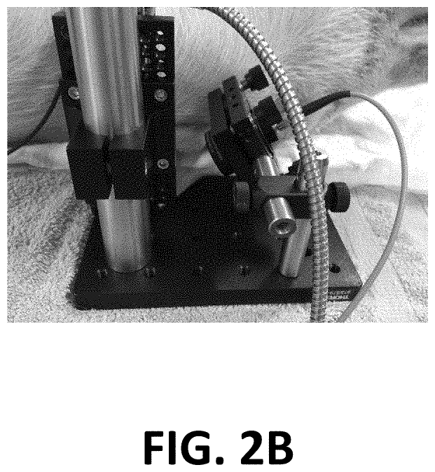

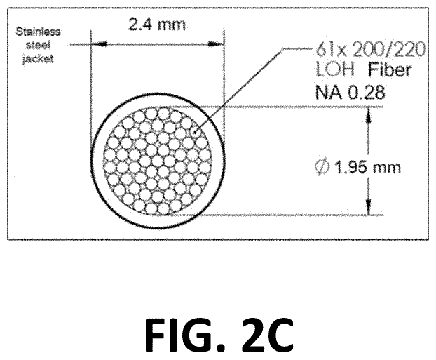

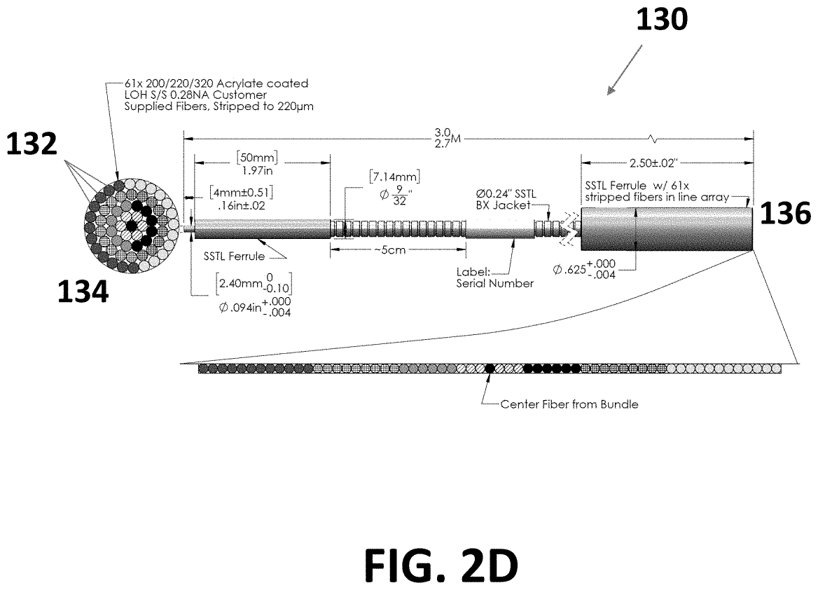

FIG. 2A shows a schematic diagram of a probe or holder that can deliver the Raman pump beam to the surface used in the Raman spectroscopy system shown in FIG. 1. The Raman pump source illuminates a filtered laser beam (e.g., 250 mW) that is focused on a target (e.g., skin on the patient's ear or forearm) with an incidence angle that is about 15 degrees to about 45 degrees from the Raman pump beam to the skin, forming an elliptical beam (e.g., 1 mm×2 mm). The area of the skin probed is laterally displaced from the spot illuminated by the Raman pump beam by up to about 3 millimeters.

FIGS. 2B and 2C show a side view and a top view, respectively, of a fiber bundle 130 in the collection optics 110 in the Raman spectroscopy system 100 in FIG. 1. Light emission from a measurement spot on the surface of the skin 108 is collected with a custom-made, round-to-linear optical fiber-bundle (e.g., LEONI Fiber Optics, Inc., VA USA with sixty-one fibers having 200 μm core diameters, Fiberguide Industries). The fiber bundle 130 has a distal end 134 (FIG. 2D) disposed about 1 millimeter to about 10 millimeters (e.g., about 5 millimeters) above the skin 108 anywhere from right above the illuminated spot to up to about 5 millimeters (e.g., 0 mm, 1 mm, 2 mm, 3 mm, 4 mm, or 5 mm) from the edge of the elliptical spot illuminated by the Raman pump beam. The fiber bundle 130 also has a proximal end 136 (FIG. 2D) in optical communication with the spectrometer 126. The fibers in the fiber bundle 130 spatially filter stray light, such as Rayleigh scattering from the surface of the skin 108, and guide Raman signal photons transmitted through the surface of the skin 108, increasing the signal-to-noise ratio (SNR) and enhancing the measurement sensitivity.

The low-pass filter 114 transmits the Raman signal photons and blocks light at the wavelength of the Raman pump beam from the detector array 122, further enhancing the SNR and sensitivity. This filter 114 can be placed at either end of the fiber bundle 130 and can also be implemented as a band-pass filter whose passband includes the Raman signal wavelength(s) but not the Raman pump beam wavelength.

Different designs for the collection optics 110 are also possible and were used in the Raman probe used in Trial 2 (described below). Instead of using a 2 mm-diameter fiber bundle directly over the sampling volume for collection of Raman photons, this alternative Raman probe was an imaging-type Raman probe with lenses for higher numerical aperture (NA) collection from skin. The magnification of the probe was set to match the diameter of the 1.95 mm-diameter input aperture of the fiber bundle.

FIG. 2D shows a side view of the fiber bundle 130 and how the fibers 132 in the fiber bundle map to the pixels in the detector array 122. The fibers 132 are arranged in concentric circles at the distal end 134 of the fiber bundle 130 and in a linear array at the proximal end 136 of the fiber bundle 130. (The fibers 132 can be arranged in other arrays as well; for example, the fibers 132 may be arranged at the proximal end 136 in a rectangular array that maps to a rectangular array of pixels in a 2D detector array.)

In this case, the detector array 122 is a two-dimensional detector array comprising at least one row or column for each fiber in the fiber bundle. The dispersive element (grating 120) spectrally disperses the Raman light from each fiber in the fiber bundle 130 along a corresponding row or column in the two-dimensional detector array 122. As a result, the intensity detected by each row of the detector array 122 represents the spectrum of the Raman light collected by the corresponding fiber. If each fiber in the fiber bundle 130 maps uniquely to a row or set of rows (or column(s)) in the detector array 122, the processor 124 can produce a Raman spectral image of the skin 108 underneath the fiber bundle 130.

Alternatively, the detector array 122 can be replaced by one or more discrete photodetectors, each of which monitors a particular spectral bin (e.g., a characteristic peak in the Raman spectrum). These photodetectors can be arranged to detect light dispersed by the grating or other dispersive element. Or the entire spectrometer can be replaced by one bandpass filter for each photodetector, with each bandpass filter transmitting light in the band monitored by the corresponding photodetector and rejecting light at other wavelengths.

The detector array 122 is configured to integrate the Raman light over a series of sequential integration periods. The length(s) and duty cycle of these integration periods depends in part on the dynamic range of the detector array 122 and the intensity of the Raman signal. For example, every five minutes (300 seconds), the detector array 122 may acquire a full-frame image for 285 seconds under the control of Lightfield software (Princeton Instruments, NJ USA). After a 15-second dead time, the detector array 122 repeats the integration. The integration periods can be between five and ten minutes long and can be separated by one hour or less.

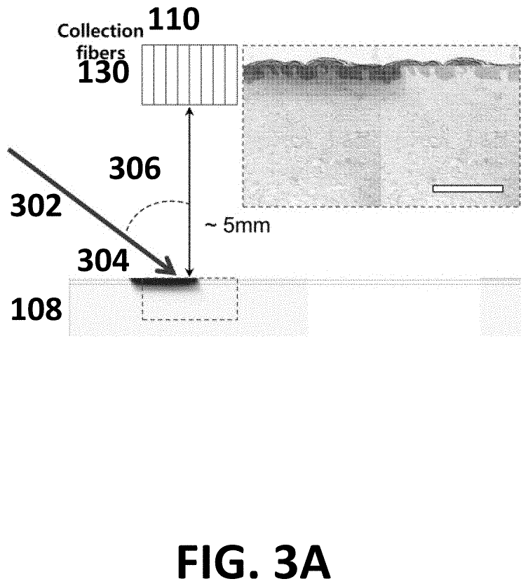

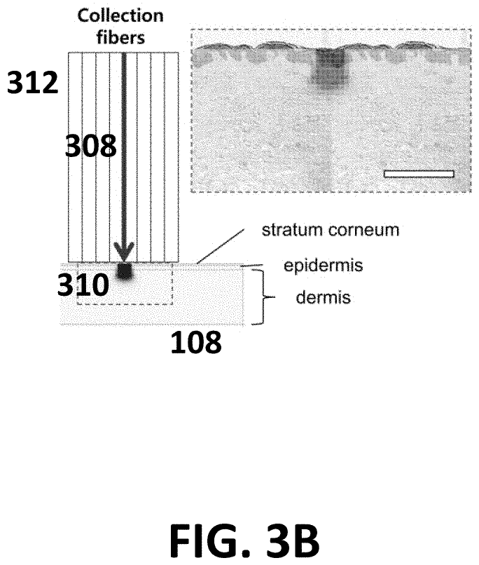

FIGS. 3A and 3B show measured radiance distributions over the sampling volume with an off-axis Raman probe like the one in FIGS. 1 and 2A and a more conventional on-axis Raman probe, respectively. In each figure, the insets show the radiance distribution overlaid on the hematoxylin and eosin stained histology images of pig ear tissue. Both insets show radiance distribution over 1 mm depth from the surface of skin.

FIG. 3A illustrates an off-axis Raman excitation and on-axis collection configuration (scale bar of 500 μm in the inset) as in the system 100 of FIG. 1. This illumination-collection geometry features oblique angle (off-axis) incidence of a laser beam 302 on a roughly elliptical spot 304 on a skin model 108 and non-contact, vertical Raman collection by the fiber bundle 130. The laser beam 302 forms an angle 306 of about 45-75 degrees (e.g., about 60 degrees) with the optical axis of the fiber bundle 130. This illumination-collection geometry gives better-spread laser radiance over the dermis layer of the skin 108, where ISF-containing glucose molecules are distributed. The closer a sampled voxel is to the fiber bundle 130, the more chance it may contribute to the Raman signal, increasing the effective sampling volume of the targeted layer while reducing the collection of background signals.

In the configuration of FIG. 3A, the Raman photons follow a trajectory in the tissue called a “banana trajectory” because it looks like a banana between source and detector. For an elliptical illuminated spot on the skin with 1 mm long minor axis and a 2 mm long major axis, the sampling depth is about 0.5 mm to about 1 mm from the skin surface. The actual sampling volume/depth also depends on the tissue absorption/scattering parameters.

FIG. 3B illustrates an on-axis Raman excitation and collection configuration in the skin model. This on-axis Raman excitation and collection represents a conventional endoscope-type Raman probe 312 touching the skin 108 (scale bar of 500 μm). A central fiber in the endoscope-type Raman probe guides the Raman pump beam 308 to a circular spot 310 on the skin 108. The other fibers in endoscope-type Raman probe 312 guide Raman signal light from the skin 108 to a detector (not shown). Compared with FIG. 3A, the illumination-collection geometry in FIG. 3B using vertical angle (on-axis) incidence of laser beam 308 and contact, vertical Raman collection by the fiber bundle 312 gives less spread and more focused distribution of radiance over the dermis layer of the skin 108, and therefore a smaller sampling volume (the region including and immediately surrounding the circular spot 310).

### Monitoring and Predicting Blood Glucose Levels

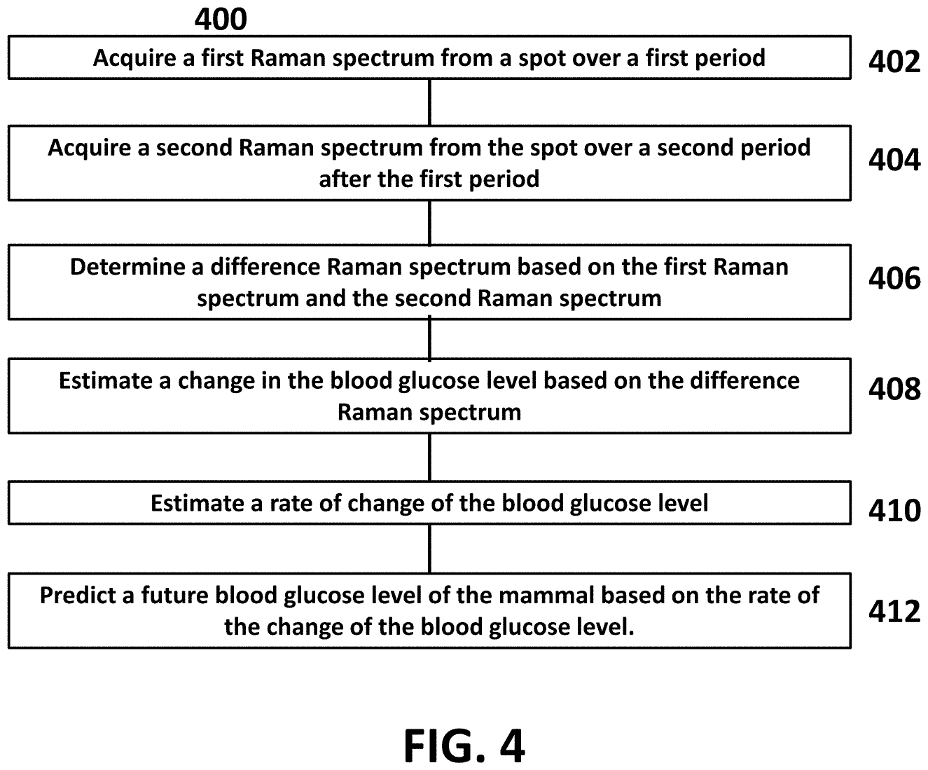

FIG. 4 shows a flow chart illustrating a method 400 of monitoring and predicting blood glucose levels. In one example, the method 400 can be used on a mammal. In 402, a first Raman spectrum is acquired from a first spot on the mammal's skin by integrating the Raman light transmitted through the first spot with a detector over a first period. In 404, a second Raman spectrum is acquired from the first spot over a second period after the first period. Both 402 and 404 may comprise illuminating a second spot on the mammal's skin with a Raman pump beam at an oblique angle (e.g., about 15-45 degrees) with respect to the mammal's skin. This illuminated second spot may be laterally displaced from the first spot by up to about 3 millimeters (e.g., 0.5 millimeters, 1.0 millimeters, 1.5 millimeters, 2.0 millimeters, 2.5 millimeters).

In 406, a difference between on the first Raman spectrum and the second Raman spectrum is determined (for example, a difference spectrum or a difference in amplitude of a peak at 1125 cm⁻¹). In 408, a change in the blood glucose level between the end of the first period and the end of the second period is estimated based on the difference Raman spectrum. In 410, a rate of change of the blood glucose level is estimated based on the difference spectrum and a duration of the second period (for example, the change in blood glucose level can be divided by the duration of the second period to give the rate of change to first order). In 412, a future blood glucose level is predicted based on the rate of the change of the blood glucose level. This predicted blood glucose level may be for a time anywhere from seconds into the future to an hour into the future (e.g., 1 minute, 5 minutes, 10 minutes, 15 minutes, 30 minutes, or 45 minutes into the future).

Equivalently, the difference spectrum can be combined with the second Raman spectrum to yield a predicted Raman spectrum, which in turn is used to generate a predicted blood glucose level based on correlations between Raman spectra and blood glucose levels.

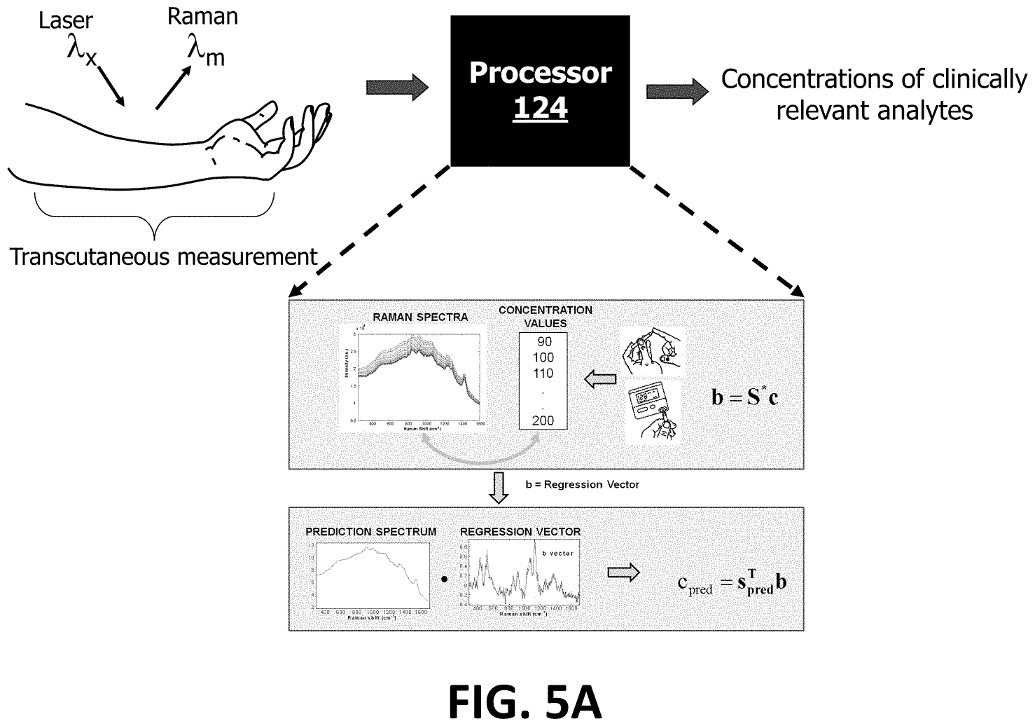

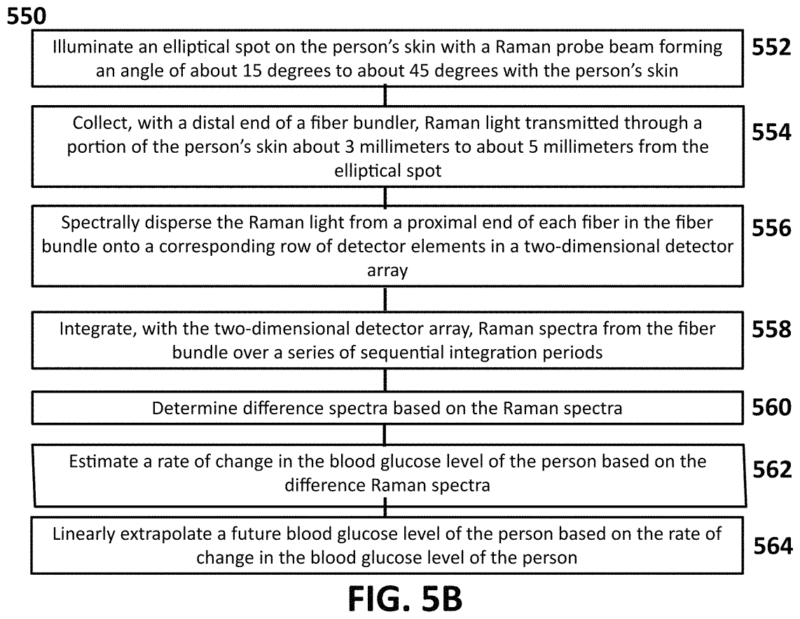

FIG. 5A shows a schematic diagram of non-invasively monitoring and predicting a blood glucose level of a person using the Raman spectroscopy system in FIG. 1. A Raman beam from a laser illuminates a spot of a person's skin, and the scatted Raman light is collected and detected to obtain Raman spectra and a predicted Raman spectrum for further analysis as described above with respect to FIGS. 1-3A. The predicted Raman spectrum can be obtained by linearly extrapolating from the measured Raman spectra as explained above with respect to FIG. 4. The measured Raman spectra are also correlated with blood glucose levels measured using conventional techniques (e.g., finger sticks). The correlation between the measured Raman spectra and the measured blood glucose levels yields a regression vector b that can be used to generate a predicted blood glucose level from the predicted Raman spectrum. (This correlation and the generation of the regression vector may take place during calibration or initial setup.) The measured and/or predicted Raman spectra combined with the measured and predicted blood-glucose concentration values and the regression vector can be used to obtain concentrations of clinically relevant analytes.

FIG. 5B illustrates a method 550 of non-invasively monitoring and predicting a blood glucose level of a person using the Raman spectroscopy system in FIG. 1. In 552, an elliptical spot on the person's skin is illuminated with a Raman probe beam forming an angle of about 15 degrees to about 45 degrees with the person's skin. In 554, the Raman light transmitted through a portion of the person's skin about 3 millimeters to about 5 millimeters from the elliptical spot is collected with a distal end of a fiber bundle. In 556, the Raman light is spectrally dispersed from a proximal end of each fiber in the fiber bundle onto a corresponding row of detector elements in a two-dimensional detector array. In 558, Raman spectra collected with the fiber bundle are integrated with the two-dimensional detector array over a series of sequential integration periods. In 560, difference spectra based on the Raman spectra is determined. In 562, a rate of change in the blood glucose level of the person based on the difference Raman spectra is estimated. In 564, a future blood glucose level of the person based on the rate of change in the blood glucose level of the person is linearly extrapolated.

### Glucose Clamping Experiments in Live Pigs

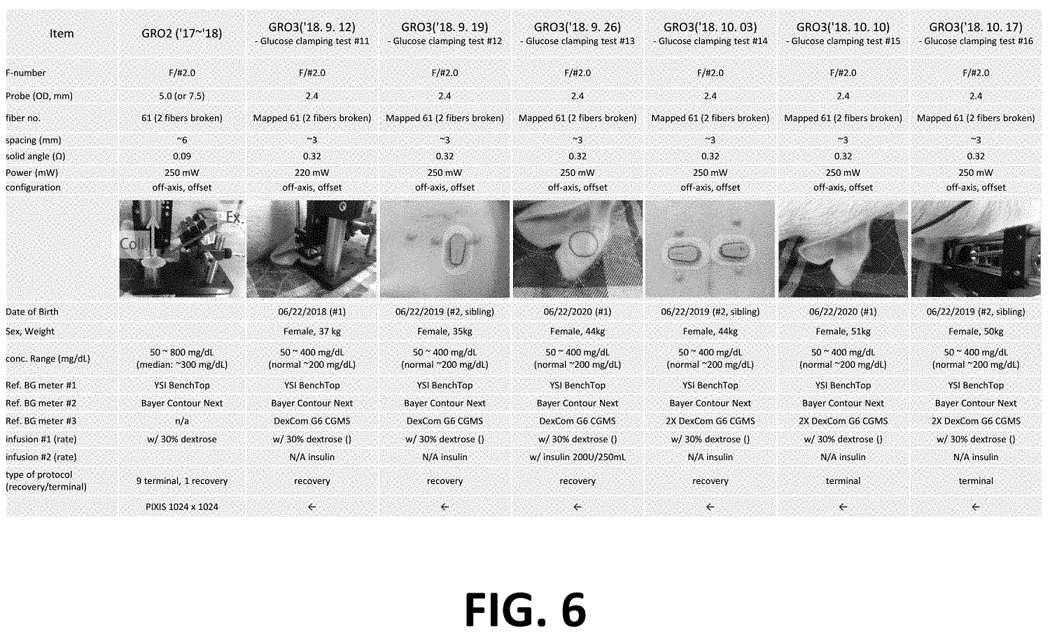

FIG. 6 illustrates glucose clamping experiments with live pigs using the systems and methods described above. In an approved animal experiment protocol, three female Yorkshire pigs (weighing between 40 kg and 55 kg) were selected for the glucose clamping test, considering anatomical and biochemical similarity. Each pig was anesthetized under 2% isoflurane supplied via a controlled vaporizer after sedation with Telazol (5 mg/kg) and xylazine (2 mg/kg) intramuscularly and given atropine 0.04 mg/kg. Each pig's anesthesia and vital signals were monitored during the experiment. Two femoral vein catheters were placed in each of the pig's leg aseptically for delivery of intravenous fluids, glucose, and repeated bleeds followed by flushing of heparinized saline (10 u/mL) between blood draws. The body temperature of swine was maintained with a heated table and water-circulating blankets. Vital signs including body temperature were examined, and no significant correlation between body temperature and glucose levels was found.

The blood glucose level was modulated within the range from 52 mg/dl to 914 mg/dl by infusing 30% dextrose and 0.8 u/ml insulin for a period of 30 to 60 minutes at each level. 3 ml of blood samples are drawn every 5 min from another catheter and were analyzed using a glucose analyzer (YSI 2300, YSI Inc., OH USA). After the measurements, the ear tissue (˜1 cm²) that was illuminated by the laser beam spot (˜1.6 mm²) was collected for histological analysis. No substantial change in the irradiated skin regions was observed under the selected power level for spectrum measurement. In the experiments, the animal was euthanized with 100 mg/kg of pentobarbital (intravenous administration of Fatal Plus). Clamping level profiles were designed to have maximum modulation and to avoid monotonic increases or decreases in reference glucose concentration as well as similar patterns between subjects, while considering clinical constraints, such as time available for the session and the recommended infusion rate depending on the subject's weight.

Because the high-throughput system equipped with the large area CCD can cause image curvature of spectrum, image curvature correction was performed for conversion from frame image to spectrum. Two consecutive spectra, each collected with an integration period of 5 minutes and a time interval of 5 minutes, were averaged into one 10-min-long spectrum, and Savitzky-Golay filtering was applied to smooth the spectrum. Other integration periods and time intervals can be used as long as the interval is longer than the integration period. Spectra can also be collected with an integration period of one minute or less than one minute without substantially compromising the signal-to-noise ratio. The analysis in this application can be based either on background-removed spectra in the range of 810 cm⁻¹ to 1650 cm⁻¹ by polynomial baseline subtraction or on band-area ratios. Band-area ratios were computed as area integrals under a background-subtracted spectrum in the selected four bands: three bands of glucose fingerprint at 911 cm⁻¹, 1060 cm⁻¹, and 1125 cm⁻¹, and one band of skin components at 1450 cm⁻¹, a peak for corresponding proteins and lipids.

Linear regression analysis was applied to train and test a mapping function from spectra or band-area ratios to corresponding glucose concentrations for calibration and prediction, respectively. A simple linear regression analysis was used for single spectrum intensities or single band-area ratios; multiple linear regression analysis was used for four band-area ratios; and partial least squares regression analysis was used for full-range background-subtracted spectra. For hold-out prospective prediction, the parameters were calibrated with training samples. Other validation schemes were also used, including four-fold cross-validation (CV) in single-subject recordings (intra-subject CV) and leave-one-subject-out cross-validation in the three subjects' recordings (inter-subject CV). In the four-fold cross-validation, single-subject recordings were split into approximately equally long and time-continuous partial recordings. Then, each time-continuous partial recording was tested by a linear regression model trained with the other three time-continuous partial recordings. In the leave-one-subject-out cross-validation, entire single-subject recordings were tested by a model trained with the other two subjects' recordings. In one example, all the recordings are tested once in cross-validation schemes.

Furthermore, the correlation coefficient R between actual and predicted glucose concentrations, mean absolute relative difference (MARD), and standard error in prediction (SEP) were calculated to quantify prediction performance with samples for testing, untouched in training (calibration). MATLAB (MathWorks, Mass. USA) running on a processor was used for the data analysis.

Advantages of the selected configuration were investigated with a raytracing simulation over multi-layered skin model (OpticStudio 15.5, Zemax, Wash. USA). A Henyey-Greenstein phase function was used to numerically simulate light scattering in tissue with optical coefficients (μs, g, and n) set differently for each layer, similar to known human cases. The number of voxels was approximately 11,000 and 4,200 for oblique-angle and normal laser illumination, respectively. More voxels were eligible for the collection of Raman signal under the oblique angle configuration. Collecting from more voxels helps averaging signal from a larger volume, improving the robustness of the measurement.

### Results and Analysis of Direct Observation on Glucose-Specific Raman Peaks

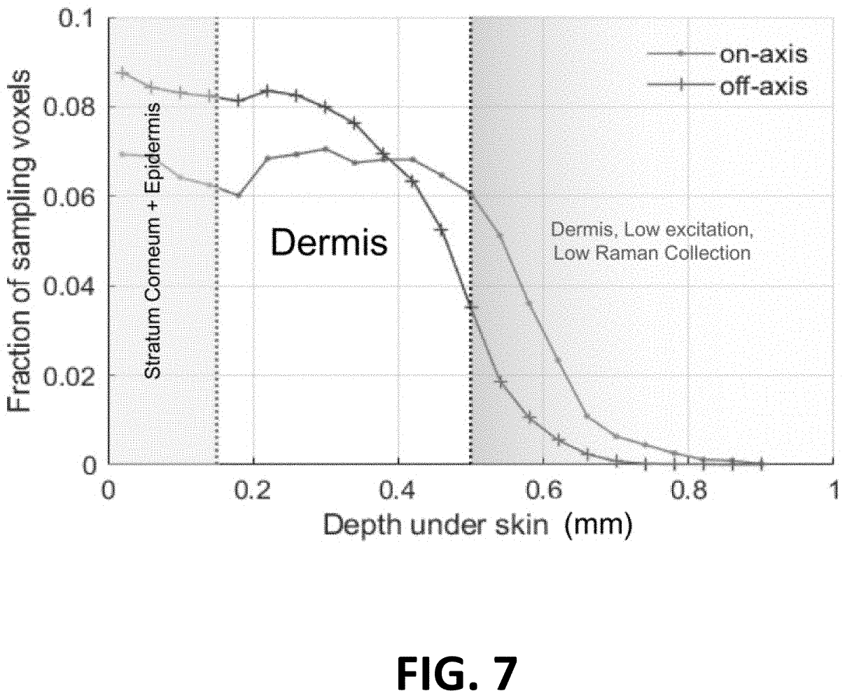

FIG. 7 shows the fraction of sampling voxels in off-axis and on-axis configurations of FIGS. 3A and 3B, respectively, as a function of depth in the confined region for the pig experiments. FIG. 7 presents the ratio of voxels at a given depth to all voxels illuminated over a certain threshold of radiance. At the dermis layer close to the fiber bundle, more voxels contribute to Raman scattering under the oblique-angle illumination than the normal illumination through a fiber. From this perspective, the oblique-angle incidence of laser can be more effective than the normal incidence in extracting a glucose Raman signal.

In one example, Raman spectra were acquired from pig ears every five minutes for approximately seven hours. The model was used on the acquired signals with four parts: glucose Raman spectrum, tissue (non-glucose) Raman spectrum, time-varying tissue background signal, and time-independent system background signal. The glucose signals varied as glucose levels were modulated during glucose clamping experiments. The non-glucose Raman spectrum mostly originated from solid skin tissue components, including lipids, proteins, and collagen. When measured from the same tissue location, the non-glucose Raman spectrum stayed relatively unchanged. Subtracting two acquired spectra with two different glucose concentrations highlights the glucose signal change.

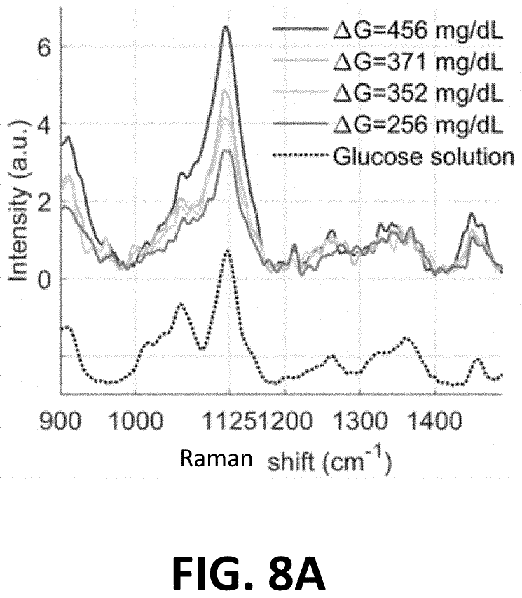

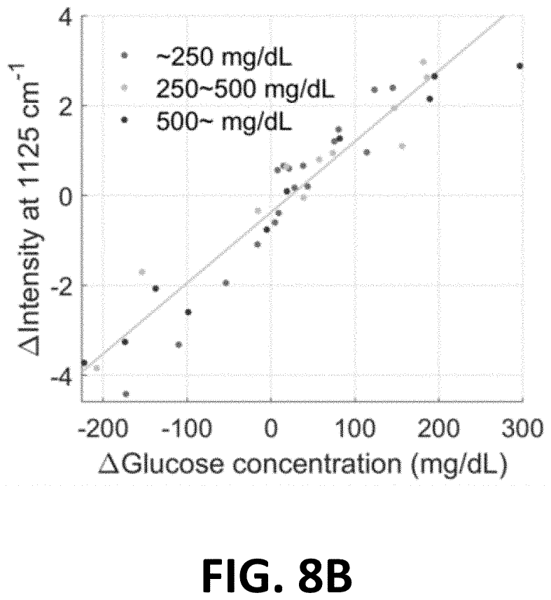

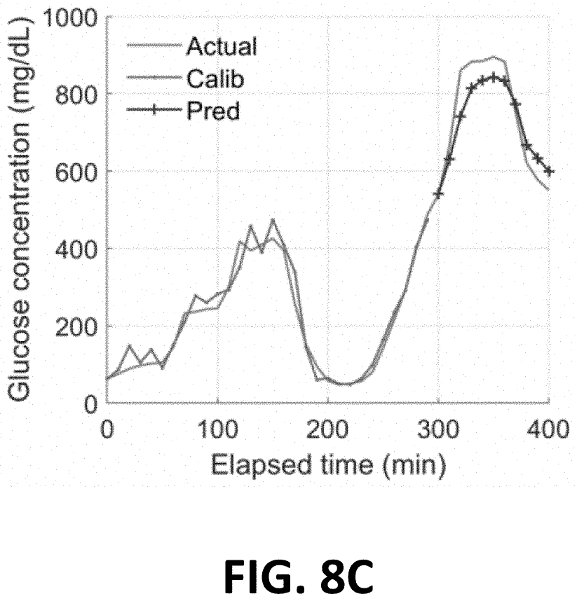

FIGS. 8A-8C show glucose Raman spectra from in vivo experiments and linearity between the spectral intensity and the corresponding blood glucose concentration. A difference spectrum between two tissue spectra with different glucose levels (G₁ and G₂) was calculated and compared to the Raman spectrum from pure glucose solution in order to demonstrate the clear glucose Raman signal from the tissue measurements.

FIG. 8A shows four subtraction spectra with four glucose differences (ΔG=G₁−G₂) along with the reference Raman spectrum from pure glucose solution. These in vivo subtraction spectra and the reference glucose solution spectrum exhibit a high degree of similarity of Pearson correlation coefficient, R=0.90 in average. One of the characteristic glucose Raman peaks appears at 1125 cm′. This peak's intensity increases linearly with the glucose difference (ΔG) increases. These results confirm that the in vivo Raman peaks indeed originate from glucose molecules in the tissue.

While FIG. 8A shows the increasing glucose intensities from four subtraction spectra, FIG. 8B shows the linear relationship between those glucose signal intensities and the corresponding glucose concentration differences during the entire measurement period. More specifically, FIG. 8B shows a linearity of R=0.95 between the change in spectral intensity and the glucose difference (ΔG) over the entire measurement time in Trial 1. The differential peak intensities were obtained by subtracting the time-moving subject-specific spectrum, lagging 20 minutes behind the spectrum to be subtracted. Three different traces represent three ranges of actual glucose concentration ranges: lower than 250 mg/dL, between 250 mg/dL and 500 mg/dL, and 500 mg/dL and higher. The linear relationship is maintained as R=0.97, 0.96, and 0.98, in the ranges lower than 250 mg/dL, between 250 mg/dL and 500 mg/dL, and 500 mg/dL and higher, respectively.

FIG. 8C shows predicted, measured, and calibrated glucose concentrations as a function of time. The predicted glucose concentration was predicted prospectively by a linear regression based on the peak intensity change and the glucose concentration difference ΔG (R=0.95 and MARD=6.6% for the prediction). The prediction can be for up to one hour. All three traces overlap significantly, indicating the high quality of the prospective prediction.

The analyses in FIGS. 8A-8C confirm the existence of the glucose signal in the acquired Raman spectra and the linear relationship of the glucose signal to the corresponding glucose concentration difference. Difference spectrum-based prediction involves two measurements at different times for one prediction. When used as a continuous glucose monitoring sensor, this condition can be achieved with periodic measurements.

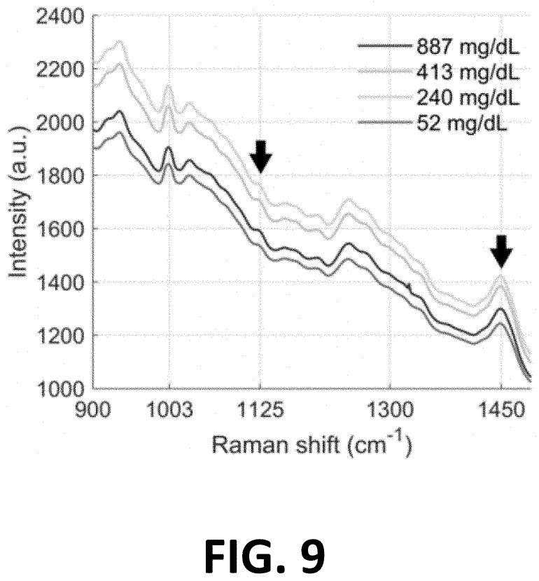

FIG. 9 illustrates the linear relationship between the band-area ratio and glucose concentration. An examination of the normalized glucose intensity, calculated as the ratio between the glucose Raman peak intensity (band) and dominant tissue Raman peak intensity (band) in a single measurement spectrum, yields a prediction from a single measurement. FIG. 9 shows averaged spectra during each of the four clamping periods. The legend shows the average glucose concentration for each clamping period. The black arrows indicate the glucose Raman peak at 1125 cm′ and the protein/lipid peak at 1450 cm′. For the tissue Raman intensity in this study, the strongest Raman band at 1450 cm′ in a spectrum was used, corresponding to the skin protein and lipid.

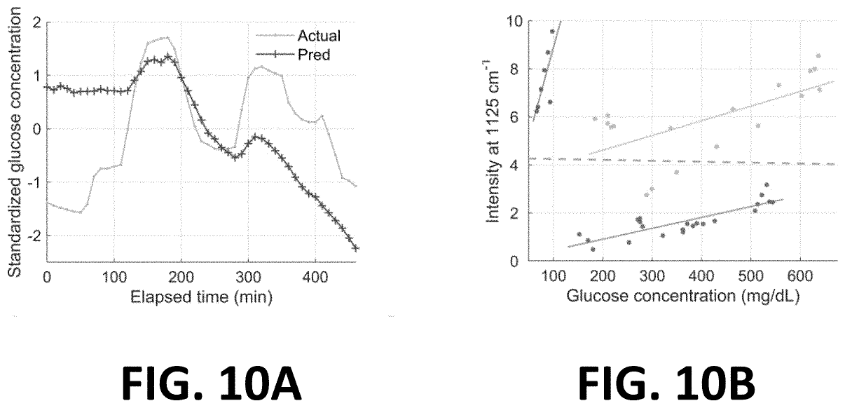

FIGS. 10A and 10B show predictions in inter-subject recordings. A model was trained with recordings from Trials 1 and 2 and used for prediction in recordings from Trial 3. FIG. 10A shows actual and predicted glucose concentrations using PLS regression with full-range background-subtracted spectra for the prediction (R=0.17). FIG. 10B shows linearity between the Raman peak intensity and glucose concentration for Trial 3 only for three partial recordings in time (separate traces, with an average R=0.74), but not for the entire recording (R=−0.02) with the spectra subtraction method.

Both the band-area method and the full-range spectrum method yielded accurate predictions in the intra-subject CV (R=0.97 and 0.98, respectively). In case of inter-subject CV or universal calibration, the band-area approach produced more accurate predictions for Trial 1 (R=0.95) than the conventional approach (R=0.87) indicating the direct glucose signal based prediction is more robust than the statistical prediction. Also, in the inter-subject CV for the other two trials, the band-area ratio method produced better results (R=0.83 in average in all the three trials) than the full-spectrum method (R=0.62 in average). The improved trend tracking capability, especially for Trial 3, can also be seen in FIG. 10A.

Close examination of the data in Trial 3 suggests that there might have been a couple of disturbances during the measurement. In FIG. 10B, despite no linear correlation for the entire recording, there is still linearity for partial recordings. External perturbations, such as movements of the subject, may have caused the abrupt changes in the background and the Raman collection efficiency. Such changes can be better corrected with the four selected band-area ratios. Because statistical learning approaches, such as PLS regression, can produce desirable outputs when similar patterns accumulate, training with full-range spectra from Trials 1 and 2 may not explain the broken linearity well in the full-range spectra from Trial 3 in the PLS regression-based full-range spectrum method.

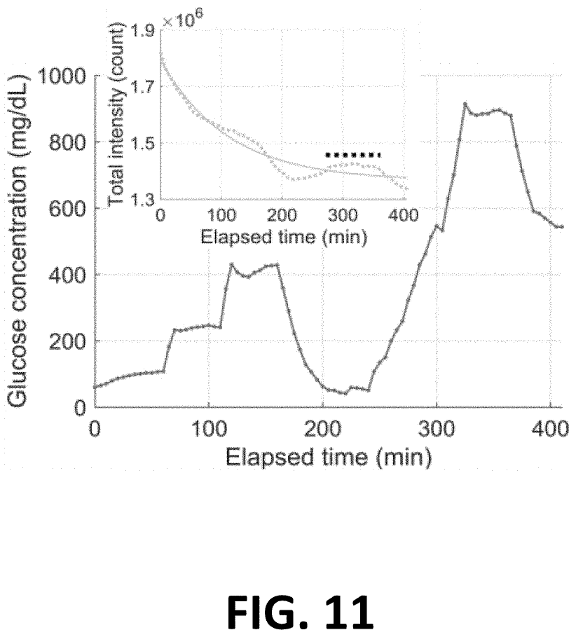

FIG. 11 shows the glucose profile during the glucose clamping experiment in Trial 1. The inset shows the exponential time decay of the fluorescence from the skin (spectrum wavelength-integration; the dotted line represents actual data and the gray line is an exponential approximation). The dotted black line in the inset indicates the time period during which fluorescence stays nearly flat.

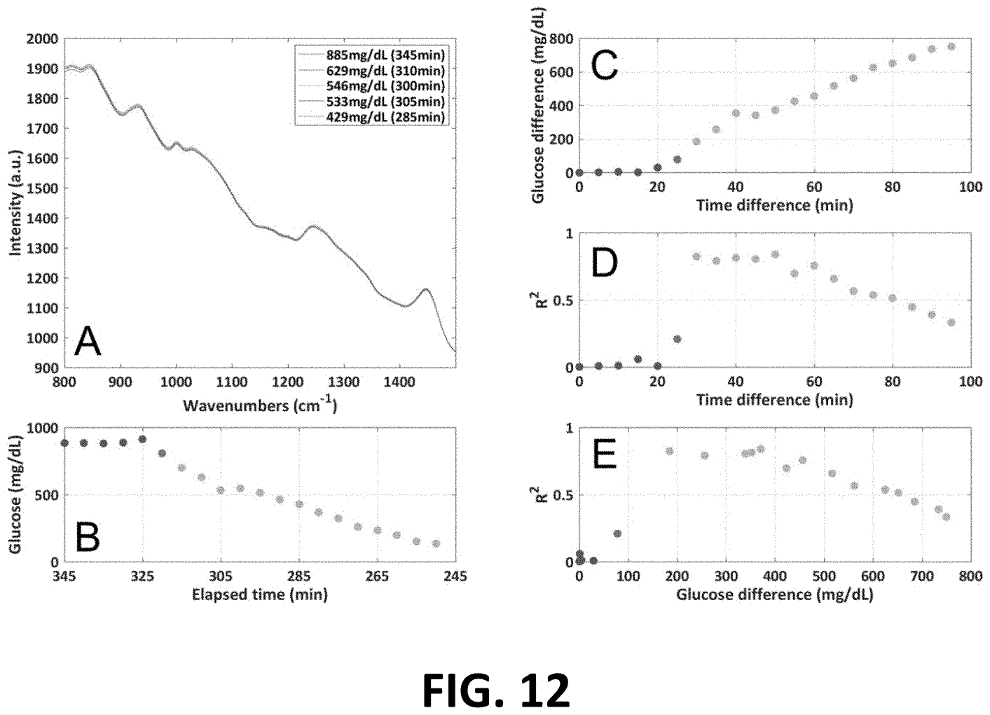

FIGS. 12A-12E shows the analysis of the validity of calibrations using the background subtraction method used in FIG. 8A and the limit of detection (LoD) measurement. FIGS. 12A-12E also illustrate the correlation coefficient trace during the time period used in the first analysis. The approach uses the correlation coefficient between the glucose solution spectrum and the difference spectra, similar to the method illustrated in FIG. 4. The smallest glucose concentration at which the corresponding Raman spectrum can appear above the noise level is investigated. Glucose signal differences in subtracted spectra are below the noise level when the corresponding glucose concentration differences are smaller than 29 mg/dL. At a glucose concentration difference of 78 mg/dL, a distinguishing correlation coefficient from the prior data can be observed. Although the exact value due to the limited data points from in vivo experiment cannot be determined, the minimum detectable concentration is estimated between 29 mg/dL and 78 mg/dL.

FIG. 12A shows raw spectra during the period when fluorescence stayed relatively flat. FIG. 12B shows glucose concentrations from the elapsed time of 345 min to an elapsed time of 250 min in a time reversal manner. FIG. 12C shows glucose concentration differences depending on the time difference from the reference at 345 min. The subtraction reference at the elapsed time of 345 min is located at the time difference of zero in FIG. 12C and FIG. 12D. The abscissa of the time difference in FIGS. 12C and 12D is plotted in a time reversal manner.

FIG. 12D shows changes in the squared Pearson correlation coefficient between the subtraction spectra and the spectrum of pure glucose in solution. This demonstrates high correlation coefficients for about 50 minutes, when enough differences started to appear in the corresponding glucose concentrations between subtraction spectra. The initial low correlations are due to the small glucose change from the approximately flat glucose level. This indicates that the calibration using the subtraction reference spectrum can stay valid and reliable for about 50 min.

FIG. 12E shows the squared Pearson correlation coefficients as a function of glucose concentration difference, combining the results in FIG. 12C and FIG. 12D. For glucose concentration differences smaller than about 30 mg/dL, glucose signal differences in the corresponding difference spectra were below the noise level. At a glucose concentration difference of 78 mg/dL, a distinguishing correlation coefficient from the prior data starts to appear. The unfilled gap with data between 30 mg/dL and 78 mg/dL is due to the limited number of datasets from the in vivo experiment.

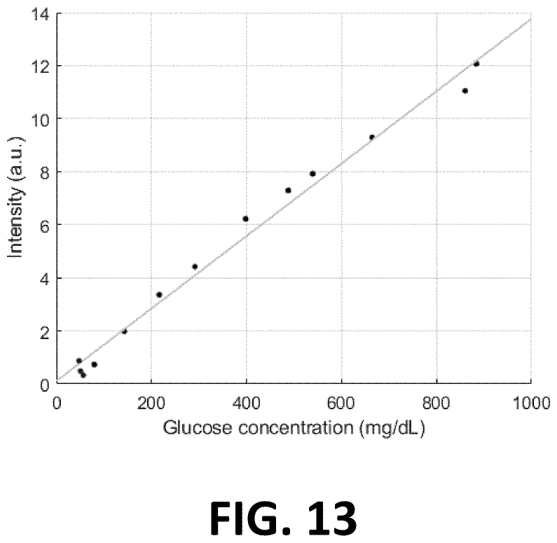

FIG. 13 shows the estimated intensity on the LoD versus glucose concentration during the time period when fluorescence stayed relatively flat, as shown in FIG. 11, using a linear regression. In order to verify our analyses with the optical system, the estimation of LoD of our system by two approaches is examined. One approach is based on linear regression. The other approach is based on the change of correlation coefficient between the difference spectrum and the reference glucose solution spectrum. If the instrument response y is linearly related to the concentration x as y=a+b·x, the LoD is defined as 3SDₐ/b, where SDₐ is the standard deviation of y-residuals, and b is the slope of the linear curve (sensitivity). Using the LoD definition with minimal glucose concentrations around 52 mg/dL, the LoD of our measurements was calculated as about 75 mg/dL. The standard deviation is calculated with the datasets nearest to zero concentration.

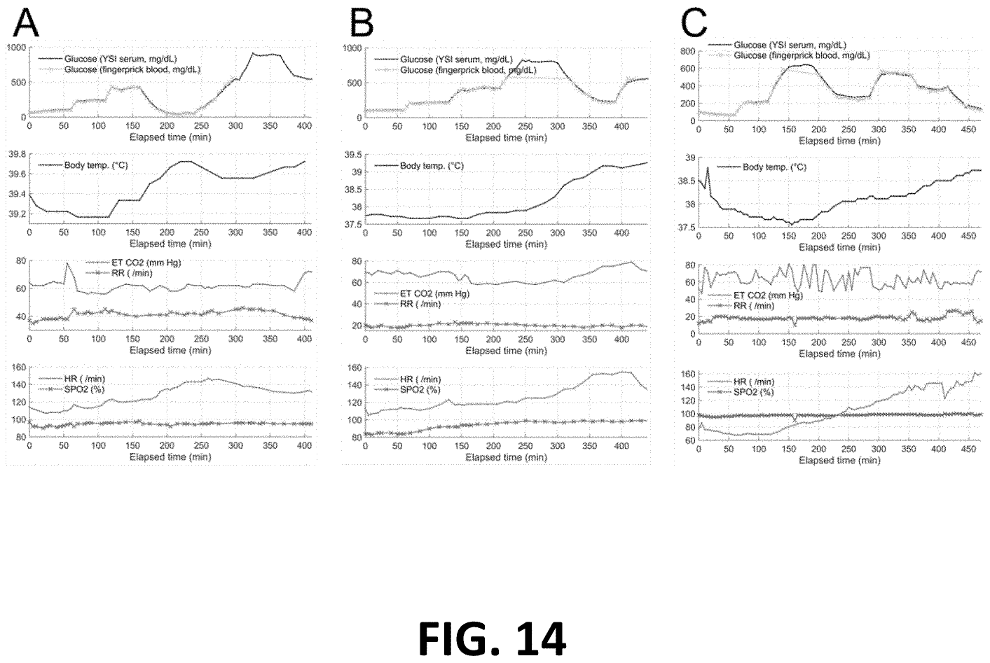

FIGS. 14A-14C show the change in glucose concentrations measured by the YSI glucose analyzer and^{AccuChek}® finger-prickers (top panels) and vital signs from the subjects in Trials 1-3, respectively. The vital signs include the subject's body temperature, end-tidal CO₂ (ET CO2), respiration rate (RR), heart rate (HR), and blood oxygen saturation (SPO2).

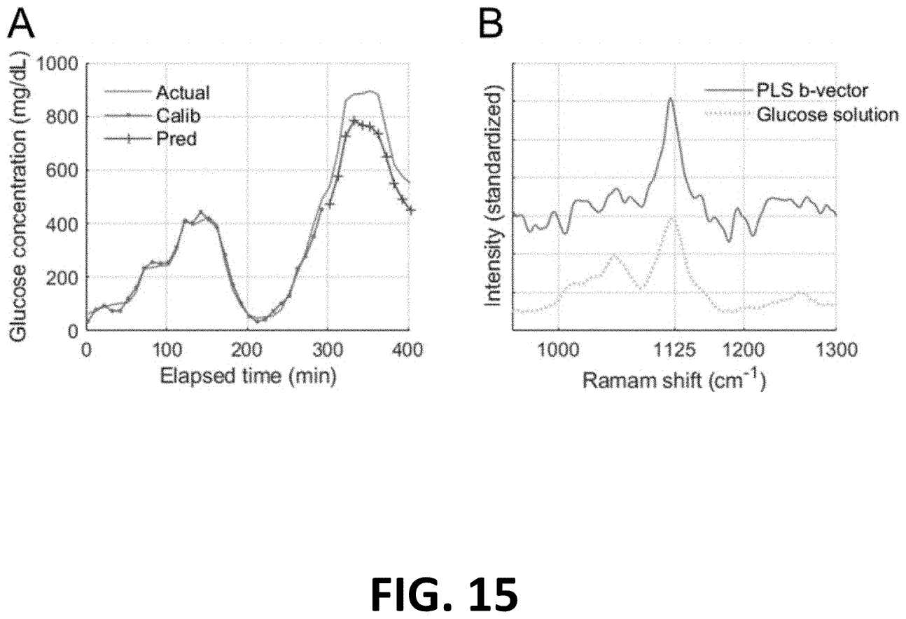

FIG. 15A shows glucose concentration prediction from a PLS regression analysis using full-range background-subtracted spectra in Trial 1. The predicted results are R=0.99 and MARD=14.1%. FIG. 15B shows that the PLSR b-vector used in FIG. 15A is comparable to glucose solution spectrum. This represents the Raman spectra capture glucose signals.

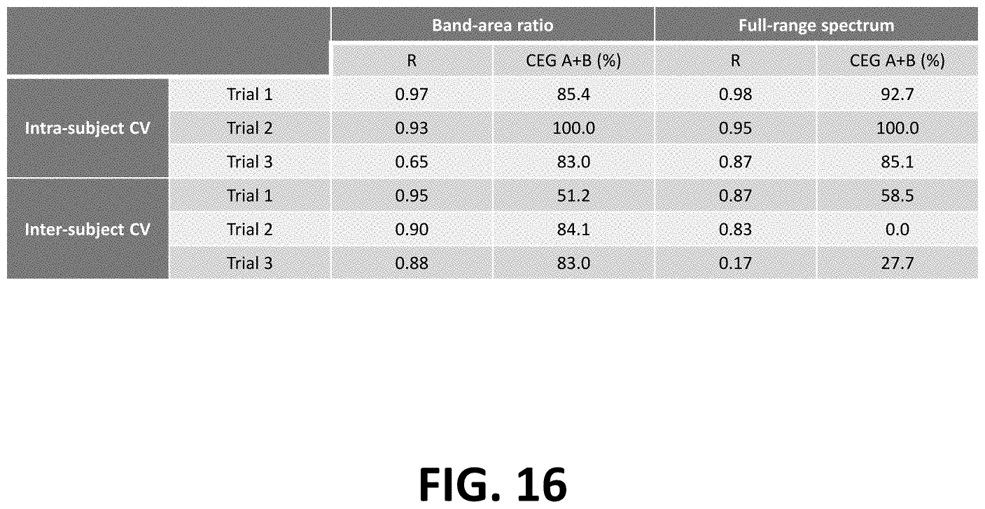

FIG. 16 summarizes the comparison results in the intra-subject CV and inter-subject CV for Trials 1-3. Four-fold cross-validation in single-subject recordings (intra-subject CV) and leave-one-subject-out cross-validation in multiple-subject recordings (inter-subject CV) were performed in order to check the feasibility of universal calibration using the direct glucose signal. For each of the cross-validation schemes, the suggested four band-area ratios with MLR and full-range spectra with PLS regression commonly used in glucose concentration prediction, were compared. MLR with four selected band-area ratios and PLS regression with full-range spectra were used. CEG refers to consensus error grid.

In addition to the identification of glucose fingerprint peaks, prospective prediction in single-subject recordings and prediction in intra-subject and inter-subject cross-validation manners were investigated. One aspect of the prediction investigation is that the analysis was performed on experiments with complex blood-glucose time-profiles. Many previous studies on non-invasive glucose sensing have claimed their possibility on glucose concentration prediction but based on relatively simple blood-glucose time-profiles, such as one on the oral glucose tolerance test with a monotonic increase and decrease in glucose concentration. However, training and testing regression with simple blood-glucose time-profiles could misdirect the regression analysis, yielding overly optimistic predictions without actual glucose sensing. Statistical learning regression modeling, such as Neural Network regression, could produce an accurate prediction when it captures, for example, an erroneous relationship between a certain non-glucose-related artifact and measurement time that is highly correlated with glucose concentration profiles, especially in simple ones. An erroneous relationship can include a time-dependent background signal or a change of the signal due to subject movement. Statistical modeling provides more robust predictions compared to a simple regression model.

As the acquired signals in our experiments include four different Raman and background signals, the following sources for signal variation can be considered. The largest signal variation comes from time-decay of auto fluorescence in in vivo skin tissue. Also, movement artifacts from an in vivo subject, even under the anesthetic state, can be another source of signal variation. When a laser-targeted spot on skin moves, the field of view of the Raman probe changes, leading to different levels of photobleaching. For the non-glucose tissue Raman spectrum, physiological changes in skin tissue during the experiment, such as sweating, may affect signal variation. Physiological vital signs from in vivo subjects, such as body temperature and heart rate, may influence the signal variation, but these experiments show no significant correlation between the intensity of the glucose fingerprint peak at 1125 cm⁻¹ (or glucose concentration) and any of the vital signs. A circulating water blanket kept the subjects' overall body temperature as stable as possible to reduce the effect of body temperature on the experiments.

The use of the intra-spectrum band-area ratio is intended to track normalized changes in glucose Raman bands using a strong band from a skin component in the same spectrum. For example, when the location of the probe or its distance to the subject's skin changes due to the subject's movement, it immediately causes a change in the intensity of the measured peaks in general. Such a change may be reflected in the entire Raman signal, including glucose Raman peaks and other skin-component Raman peaks as well. The use of the band-area ratio between the two selected bands may reduce the influence of these measurement artifacts on glucose Raman peaks by the intra-spectrum band normalization. In this sense, the band-area ratio approach can be valid, though the signal origins of glucose fingerprint peaks and the protein/lipid peak differ.

Glucose Clamping Experiments in Live Pigs Using a Physiological Blood Glucose Range

Additional glucose clamping experiments with live pigs were conducted using a blood glucose range more physiologically relevant to humans. These experiments used the same experimental conditions as described above. In an approved animal experiment protocol, three female Yorkshire pigs (weighing between 40 kg and 55 kg) were selected for the glucose clamping test, considering anatomical and biochemical similarity.

The blood glucose level was modulated within the range from about 50 mg/dl to about 400 mg/dl by infusing 30% dextrose and 0.8 u/ml insulin for a period of 30 to 60 minutes at each level. In addition to measure blood glucose with the off-axis Raman spectroscopy system, blood glucose was also measured using three reference blood glucose analyzers. Two ex situ reference blood glucose analyzers, a YSI 2300 and a Roche Accu-Chek meter, measured blood glucose in blood samples drawn every 5 minutes. The third reference blood glucose analyzer, a DexCom G6, was a minimally invasive continuous glucose monitor system (CGMS) that measured blood glucose in situ.

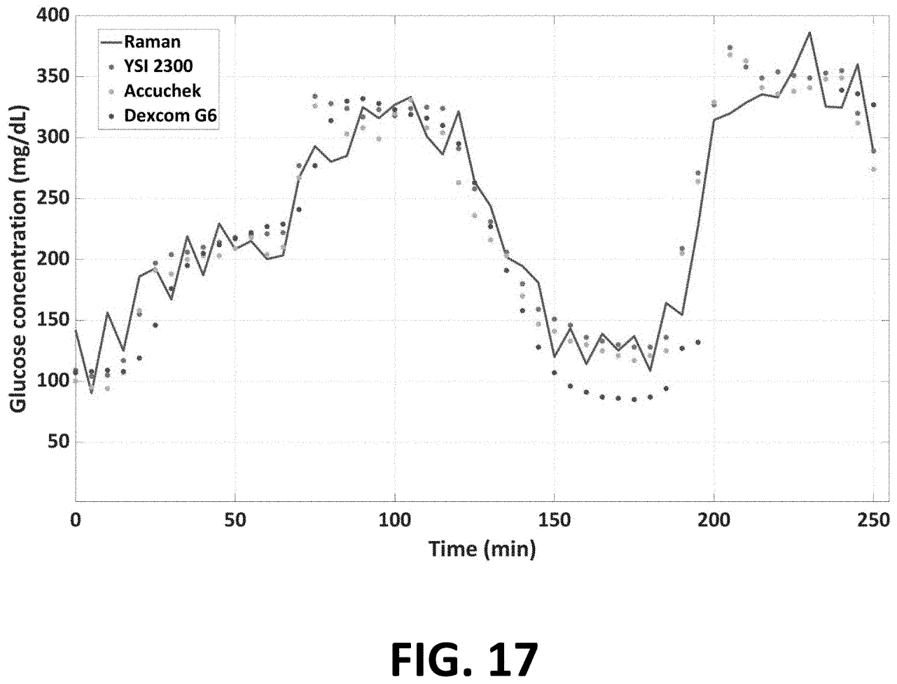

FIG. 17 shows glucose concentrations measured with the off-axis Raman spectroscopy system during a glucose clamping experiment. In addition, FIG. 17 shows glucose concentrations measured in the same time intervals with the three reference glucose analyzers described above. Similar trends in glucose concentration are given in all four traces. The figure indicates that the accuracy of the Raman spectroscopy system is on par with the accuracy of the reference glucose analyzers in measuring blood glucose in vivo within a physiologically relevant range.

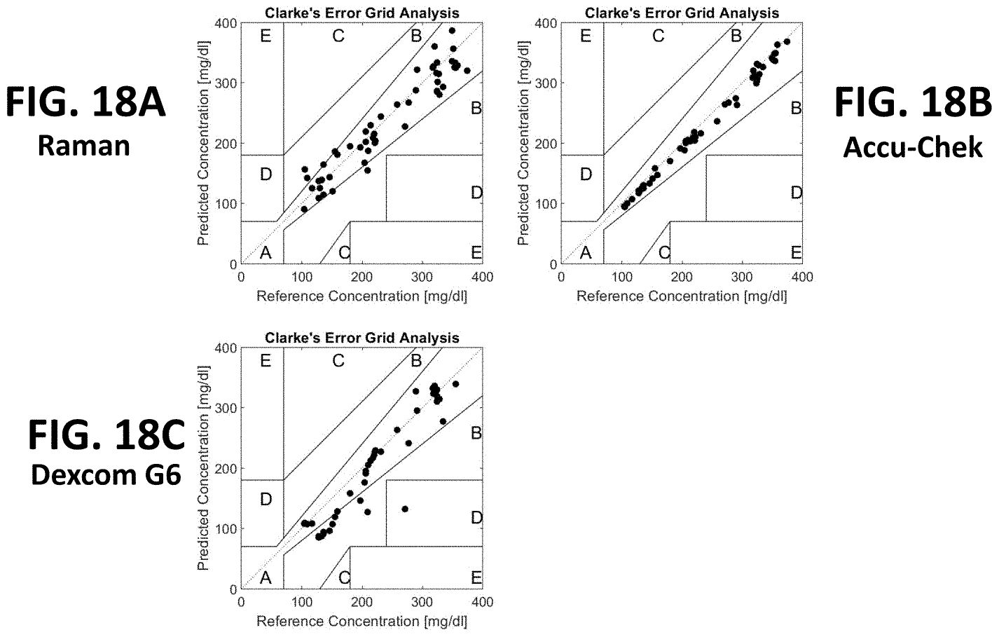

FIGS. 18A-18C show Clarke's Error Grid Analyses of the accuracy of the Raman spectroscopy system, the Accu-Chek meter, and the Dexcom G6 CGMS, respectively, in measuring blood glucose during the glucose clamping experiments. FIG. 18A shows that most of the values measured with the Raman spectroscopy system fell within 20% of the reference sensor YSI 2300 (Region A), with a few data points falling outside of this range but not in a range that would lead to inappropriate treatment (Region B). FIG. 18B shows that all of the values measured with the Accu-Chek meter fell within Region A. FIG. 18C shows that some of the values measured with the Dexcom G6 CGMS fell within Region B, and one data point fell within a range indicating a potentially dangerous failure to detect hypoglycemia or hyperglycemia (Region D). By these metrics, the Raman spectroscopy system shows greater accuracy than the Dexcom G6 CGMS in the physiological conditions tested.

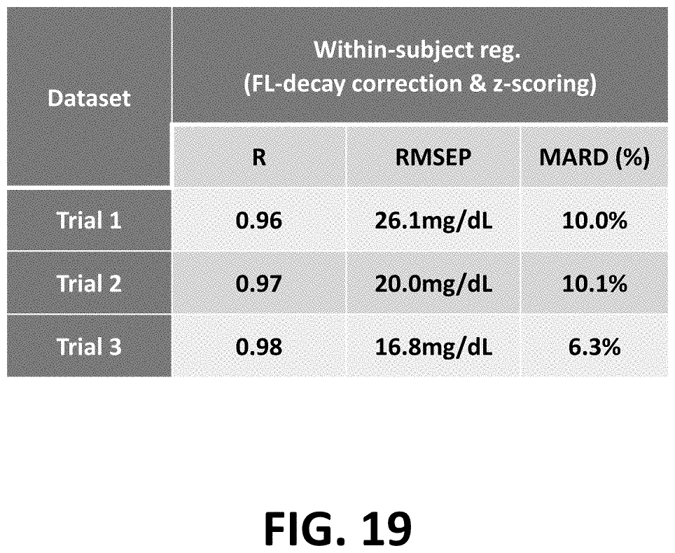

FIG. 19 summarizes the correlation coefficient R between actual and predicted glucose concentrations, root mean square error predictions (RMSEPs), and MARD values calculated from the three glucose clamping trials described above. These results indicate that glucose peak intensity measured with the Raman spectroscopy system can be used to predict glucose concentration.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. (canceled)

2. (canceled)

3. (canceled)

4. (canceled)

5. (canceled)

6. (canceled)

7. (canceled)

8. (canceled)

9. (canceled)

10. A system for non-invasively monitoring a blood glucose level of a mammal, the system comprising:
a Raman pump source to illuminate a spot on the mammal's skin with a Raman pump beam incident on the mammal's skin at an oblique angle;
collection optics to collect Raman light scattered through an area of the mammal's skin laterally displaced from the spot illuminated by the Raman pump beam; and
a detector array, in optical communication with the collection optics, to detect the Raman light collected by the collection optics, the Raman light representing the blood glucose level of the mammal,
wherein the collection optics comprises a fiber bundle having a distal end disposed about 3 millimeters to about 5 millimeters from the mammal's skin and a proximal end in optical communication with the detector array,
wherein the detector array is a two-dimensional detector array comprising at least one row for each fiber in the fiber bundle and further comprising:
a dispersive element, in optical communication with the proximal end of the fiber bundle and the two-dimensional detector array, to spectrally disperse the Raman light from each fiber along a corresponding row in the two-dimensional detector array.

11. (canceled)

12. (canceled)

13. The system of claim 10, wherein the detector array is configured to integrate the Raman light over a series of sequential integration periods.

14. The system of claim 13, further comprising:
a processor, operably coupled to the detector, to determine at least one difference spectrum based on the Raman light integrated by the detector array over the series of sequential integration periods and to estimate a change in the blood glucose level over at least one of the series of sequential integration periods based on the at least one difference spectrum.

15. The system of claim 14, wherein the processor is configured to estimate a rate of change of the blood glucose level based on the at least one difference spectrum.

16. The system of claim 10, wherein the area of the mammal's skin is laterally displaced from the spot illuminated by the Raman pump beam by up to about 3 millimeters.

17. The system of claim 10, further comprising:
a filter, in optical communication with the collection optics, to transmit the Raman light to the detector array and to block light at a wavelength of the Raman pump beam from the detector array.

18. A method of non-invasively monitoring a blood glucose level of a person, the method comprising:
illuminating an elliptical spot on the person's skin with a Raman probe beam forming an angle of about 15 degrees to about 45 degrees with the person's skin;
collecting, with a distal end of a fiber bundle, Raman light transmitted through a portion of the person's skin about 3 millimeters to about 5 millimeters from the elliptical spot;
spectrally dispersing the Raman light from a proximal end of each fiber in the fiber bundle onto a detector array;
integrating, with the detector array, Raman spectra from the fiber bundle over a series of sequential integration periods;
determining difference spectra based on the Raman spectra; and
estimating a rate of change in the blood glucose level of the person based on the difference Raman spectra.

19. The method of claim 18, wherein the detector array is a two-dimensional detector array, and wherein spectrally dispersing the Raman light comprises:
spectrally dispersing light from a fiber in the fiber bundle onto a row of detector elements in two-dimensional detector array.

20. The method of claim 18, further comprising:
linearly extrapolating a future blood glucose level of the person based on the rate of change in the blood glucose level of the person.

21. The method of claim 18, wherein determining the difference spectra comprises determining a difference in an amplitude of a peak appearing in the first Raman spectrum and a corresponding peak in the second Raman spectrum.

22. The method of claim 18, further comprising:
predicting a future blood glucose level of the mammal based on the rate of change of the blood glucose level of the mammal.
